(19) United States
(12) Patent Application Publication
(10) Pub. No.: US 2024/0066308 A1
(43) Pub. Date: Feb. 29, 2024

(54) DIRECTED WIRELESS COMMUNICATION FOR NEUROMUSCULAR ELECTRICAL STIMULATION

(71) Applicant: NMES Group AB, Svenljunga (SE)

(72) Inventors: Vincent Tellenbach, Sion Valais (CH); Vedran Stankovic, Svenljunga (CH)

(73) Assignee: NMES Group AB, Svenljunga (SE)

(21) Appl. No.: 18/272,296

(22) PCT Filed: Jan. 13, 2022
(86) PCT No.: PCT/EP2022/050690

§ 371 Date: Jul. 13, 2023

Related U.S. Application Data
(60) Provisional application No. 63/199,618, filed on Jan. 13, 2021.

(51) Int. Cl. A61N 1/372; A61N 1/04

(52) U.S. Cl. CPC A61N 1/3727; A61N 1/0484; A61N 1/37217; A61N 1/0452; A61N 1/0456

(57) ABSTRACT

A method and apparatus to deliver electrical stimulation using wireless controlled neuromuscular electrical stimulation devices are disclosed. The electrical neuromuscular stimulation device allows the delivery of short electrical impulses triggered and controlled by transmitters directed towards a receiver. The proposed method and apparatus may enable the recreation of realistic haptic sensory feedback. Possible uses include, but are not limited to gaming, entertainment, learning, training, medical, rehabilitation, and educational purposes.

### FIELD OF THE INVENTION

The present invention relates, in general, to directional wireless control of electrical stimulation devices. In particular for providing electrical stimulation of muscles and nerves for the purpose of contracting muscle groups or inducing a nervous system response.

### BACKGROUND

The number of medical applications that use electrical stimulation is large and covers virtually every living body component. These applications include prevention of muscle atrophy, promotion of wound healing, prevention of venous thrombosis, alleviation of both chronic/acute pain and prevention of incontinence to name but a few. Electrical stimulation may also be used for such non-medical objectives as muscle training, muscle toning, improving muscle endurance, and muscle relaxation.

Electrical stimulation of muscles and nerves is well established in medicine and physical therapy with a history dating back to mid-1850; such stimulation is currently achieved by applying electrodes to: 1) the skin at the point(s) of desired electrical stimulation; 2) through insertion of electrical probes into body cavities, and; 3) through surgical insertion of electrodes.

Neuromuscular Electrical Stimulation Principle

Muscle contractions are produced and controlled by the brain by means of electrical signals transmitted through the nervous system. When an electrical signal from the brain reaches the muscle, the latter is activated into groups of “motor units”, each made up of a single neuron and of a group of associated muscle cells connected to it. This initiates a chemical reaction which causes the cells in this motor unit to contract. The complete contraction of the muscle usually involves a number of motor units simultaneously, and its strength is directly proportional to the number of activated motor units. The gradual enrolment process of the motor units which consents to a perfectly controlled and smooth muscle contraction is called spatial summation.

Electrical muscle stimulation (EMS), also known as neuromuscular electrical stimulation (NMES) or electromyostimulation, is the elicitation of muscle contraction using electric impulses. The impulses are generated by a device and are delivered through electrodes on the skin near to the muscles being stimulated. The electrodes are generally pads that adhere to the skin. The impulses mimic the action potential that comes from the central nervous system, causing the muscles to contract.

When a sufficiently intense single electrical impulse reaches the motor muscle or nerve, it causes one short single contraction of the muscle (spasm). If this single spasm is repeated and the frequency of reiteration exceeds ten spasms p.s., each following spasm is enhanced by one degree of muscle shortening caused by the preceding spasm. Such an effect is called temporal summation. The lowest stimulation frequency, where the successive contractions merge, is called tetanization frequency.

Devices for providing NMES are often controlled by a wired connection to a control unit into which stimulation programs can be pre-programmed and/or user selected. Such control units may have ports for wires which connect to stimulation pads, and thus suffer from certain drawbacks in some applications.

Haptic Feedback

Haptic sensory feedback is the use of different actuators to create a realistic sensory response in order to enhance a user's experience in a virtual environment. Applications using haptic sensory feedback are very broad and cover among other things video gaming, entertainment, learning, physical training and clinical applications. Haptic sensory feedback is broadly defined as the sense of touch and includes vibration, texture, temperature, pain, force and proprioception sensations.

There is a need for improved devices for NMES, in particular in those which may be controlled to apply haptic feedback to a user.

### SUMMARY

The invention is defined by the appended independent claims. Embodiments of the invention are defined in the dependent claims.

In a first aspect, there is provided a device for providing electrical stimulation to a user, comprising: a conductive medium configured to apply electrical stimulation to a user; a receiver, configured to receive a directed wireless signal; and a processor, configured to: interpret the received signal; and initiate electrical stimulation via the conductive medium based on the interpreted signal. The receiver may be configured to receive signals from certain directions and not from others.

In this way, an NMES device can be controlled wirelessly, and from only a specific direction. This is advantageous for a number of reasons. In a medical setting, for example, it may be advantageous to initiate an electrical stimulation in specific locations, as could be determined by a medical professional operating a transmitter, or by a user in response to their felt need for stimulation. If a number of conductive media are connected to a user, it may be advantageous to stimulate only one of the media at a time, in order to provide treatment in a specific location, or provide treatment to muscles in a particular order, as this may be clinically more effective. A directionally limited wireless receiver is particularly advantageous for this purpose, because treatment can be changed (by directing the wireless transmitter) without the need to interact with a user interface on a control unit, for example. Adjusting treatment, for example for rehabilitation, can therefore be faster and more effective.

In recreational settings, directionally limited wireless receivers may also be advantageous. When gaming in a virtual or augmented reality environment, for example, it may be advantageous that electrical stimulation is only applied when the receiver is directed towards a source of wireless communication. For example, a wireless transmitter could be positioned underneath a monitor or in any other specific location. A user who, while playing a video game and with a number of conductive media placed on their body, angles their body towards the monitor and is thus angled towards the transmitter, could experience an impact only on the side of their body facing the monitor. The immersion in the gaming experience could thus be enhanced.

It will be appreciated that a receiver may be configured to receive a directed wireless signal in a number of ways. For example, the receiver itself may be configured only to register and acknowledge a signal directly incident thereon. Additionally or alternatively, the receiver may include a housing to block signals incident from an angle above a certain angular threshold.

The conductive medium may be embedded within a garment, optionally wherein the garment comprises a strap, sleeve, wrap, or item of clothing, and may comprise an electrode, optionally a carbon electrode or a hydrogel electrode.

It will also be appreciated that interpreting the received signal, as performed by the processor, may comprise any form of demodulation and/or decoding as may be necessary depending on the form of the transmitted signal. Indeed, determining that no demodulation and/or decoding is necessary is encompassed within interpreting the received signal. Interpreting the signal to derive the electrical stimulation to be provided to the user may be necessary when the signal has been modulated and/or encoded.

In this way, the device may provide an intuitive and easy to apply conductive medium. In a medical setting, for example, this may be advantageous because a garment could be designed to be worn day-to-day, such that short treatments not requiring professional medical assistance could be applied by the user at very short notice, without the need to apply dedicated conductive pads. In a recreational setting, the processor comprised within the NMES device may store a known location on the body of a conductive medium embedded within a garment, and apply electrical stimulation accordingly. For example, a simulated impact on an arm could receive a different stimulation than a simulated impact on a leg.

The conductive medium may be configured to apply an electrical stimulation causing a haptic sensation in the user and/or the interpreted signal may be configured to instruct an electrical stimulation causing a haptic sensation in the user.

The receiver may be configured to receive the directed wireless signal only when the receiver has direct line-of-sight of a transmitter of the directed wireless signal. This may be advantageous both for medical and for recreational applications. For example, if a conductive medium is embedded within a garment, a direct line-of-sight requirement may be advantageous to avoid or limit accidental stimulation. Recreationally, this may be advantageous for certain gaming applications, for example for simulating weapon use and impacts.

In a second aspect, there is provided a device for transmitting an electrical stimulation program to a device configured to provide electrical stimulation to a user, the transmission device comprising: a processor, configured to encode an electrical stimulation program into a wireless signal; and a directional transmitter, configured to transmit the signal in one or more specific directions and not in other directions.

In this way, similarly to limiting the reception of wireless signals to certain directions as described in respect of the first aspect, by introducing directionality to a wireless transmitter for electrical stimulation, both medical and recreational applications can be improved. In a medical setting, medical professionals or the user can provide targeted treatment to specific muscles without the need for pre-programming stimulation programs. Recreationally, applications which benefit from directional transmission include, but are not limited to, weapons based video gaming and/or virtual/augmented reality environments, educational and learning purposes, and training purposes. For example, a personal trainer may wish to highlight the ideal progression of muscle contraction during a certain movement in order that the user more safely perform the movement when using weight. A directional transmitter, and optionally directionally limited receivers, could be used to apply said ideal contractions accurately and with timing controlled by the personal trainer specific to the user's need.

Encoding the electrical stimulation program into a wireless signal may be advantageous to limit ambient noise, for example in a bright environment if the wireless signal uses visible light, and/or to limit crosstalk from transmitters not intended to apply electrical stimulation to the user.

Encoding the electrical stimulation program into the signal may comprise: receiving the electrical stimulation program; applying an communication protocol to the electrical stimulation program using pulse width modulation. Pulse width modulation has been found to be a particularly effective way to limit crosstalk and ambient noise.

Applying a communication protocol to the electrical stimulation program using pulse width modulation may comprise: selecting a carrier frequency known to both the device for transmitting the electrical stimulation program and to the device configured to provide electrical stimulation to a user; selecting a start pulse width and an end pulse width known to both the device for transmitting the electrical stimulation program and to the device configured to provide electrical stimulation to a user, wherein the start and end pulse widths are not equal; selecting a first pulse width to define a bit value of one and a second pulse width to define a bit value of zero, wherein the first and second pulse widths are not equal to one another or to the start and end pulse widths; converting the bits of the electrical stimulation program into pulses; concatenating the start and end pulses to the beginning and end, respectively, of the converted electrical stimulation program; and applying the carrier frequency to the concatenated pulses.

The electrical stimulation program may comprise at least one of: an identifier for the device for providing electrical stimulation to a user, an identifier for transmitting an electrical stimulation program, a length of an electrical stimulation, an intensity of electrical stimulation, a type of electrical stimulation, and a delay after reception of the signal before electrical stimulation begins. In this way, a broad range of electrical stimulation programs can be transmitted and applied to the user. Stimulation programs may differ based on the intended application, for example medical or recreational, and on the needs of the user.

The electrical stimulation program may be configured to instruct an electrical stimulation causing a haptic sensation in the user. Provision of a haptic sensation may be advantageous for a number both medical and recreational purposes, not least that the provision of controlled haptic feedback may provide a user with an enhanced realistic sensory experience.

In a third aspect, there is provided a system, comprising: a first device for providing electrical stimulation to a user according to the first aspect; a second device for transmitting an electrical stimulation program according to the second aspect. It will be appreciated that the third aspect is advantageous for the same reasons as the first and second aspects.

The system may further comprise: a third device, in communication with the first and second devices via a wireless communications protocol, the third device comprising a processor configured to: receive, from the first device, data relating to the electrical stimulation applied by the conductive medium; and receive, from the second device, data relating to the transmitted signal. The third device may also be configured to transmit data to the first and/or second devices. In other words, the wireless communications protocol may be two-way.

The third device may be configured to transmit an electrical stimulation program to the first device. In such embodiments, the transmitter device may act solely as a trigger to begin electrical stimulation according to the program transmitted by the third device. The method of communication between transmitter and receiver may be the same in such embodiments, but the encoded information transmitted simply instructs beginning and, optionally, end of stimulation.

In this way, the wireless signals passing from transmitter to receiver can be recorded, analysed, and reviewed on a third device. This may be advantageous, for example, in a recreational application, if a personal computer is being used to play a video game, the video game providing sensory inputs which are required to be translated into electrical stimulation for the user. For example, if the user were playing a first person contact sport video game on a personal computer or console, the personal computer or console may instruct the first device to transmit a wireless signal towards the user on contact with the user's avatar in the video game.

The wireless signal may be transmitted in a particular direction, or may be received by only some receivers facing a particular direction, and an electrical stimulation simulating contact may be applied to only those portions of the user's body facing that direction.

In a medical setting, for example, the third device may be a mobile device and may be configured to store and log treatment programs undergone by a particular user.

The third device may comprise a display and may be configured to record and display the data relating to the transmitted signal and/or the data relating to the electrical stimulation applied by the conductive medium. A display may be advantageous so that a user can more easily review past electrical stimulations delivered via the system. Achieving this ease of review without the need for a display on either the transmitting or receiving devices themselves allows these devices to remain smaller and more compact—increasing the number of suitable uses and reducing discomfort when worn. Additionally, a display comprised within either the transmitting or the receiving device would have power requirements requiring a larger power source (e.g. battery). Since these devices are likely to be worn or held, increasing the power source size, and therefore weight, could reduce the useability of the device. By communicating with a third device, perhaps already comprising a display, the transmitting and receiving devices can remain advantageously compact.

The wireless communications protocol employed to allow communication between the first and second devices and the third device may be Bluetooth Low Energy. Bluetooth Low Energy has been found to be a particularly efficient communication protocol for this application. In particular, it is energy efficient (again aiding the compactness of all devices) and can be used to synchronise multiple wireless NMES devices with one another.

In a fourth aspect, there is provided a method for providing electrical stimulation to a user, comprising: transmitting an electrical stimulation program from a device according to the second aspect; receiving the electrical stimulation program at a device according to the first aspect; and providing electrical stimulation to the user according to the received electrical stimulation program.

In a fifth aspect, there is provided a method for providing electrical stimulation to a user, comprising operating the system according to the fourth aspect.

In a sixth aspect, there is provided a computer-implemented method for encoding an electrical stimulation program into an encoded wireless signal, comprising: selecting a carrier frequency; selecting a start pulse width and an end pulse width, wherein the start and end pulse widths are not equal; selecting a first pulse width to define a bit value of one and a second pulse width to define a bit value of zero, wherein the first and second pulse widths are not equal to one another or to the start and end pulse widths; converting the bits of the electrical stimulation program into pulses; concatenating the start and end pulses to the beginning and end, respectively, of the converted electrical stimulation program; and applying the carrier frequency to the concatenated pulses.

It will be appreciated that methods as described herein are advantageous for the same reasons as the devices and systems performing said methods.

The directed wireless signal may be a directed infrared (IR) signal. As will be described in further detail herein, infrared is particularly advantageous as a method of communication because it is directional, has short transmission range, requires line-of-sight, and can easily be encoded. The benefits of directional and line-of-sight communication for the devices, systems, and methods of the invention are described above. In addition, these characteristics of IR are such that interception of communications is more difficult than with other wavelengths. Furthermore, and advantageously, IR transmitters and receivers are relatively compact and cheap to manufacture.

It will be appreciated that although only one receiver and transmitter have been described, devices according to the present invention may comprise a plurality of receivers and transmitters, connected to one or more processors and one or more conductive media. Similarly, devices which comprise both receivers and transmitters, and operate as such, are contemplated and are embodiments of the invention. By way of example only, a device comprising one transmitter, a plurality of receivers, three conductive media per receiver, and one processor controlling both transmitter and receivers, is an embodiment of the invention. It will also be appreciated that any or all of the devices described herein can be powered by a power supply, such as a replaceable or rechargeable battery, or could be connected to mains power, or may have both capabilities.

Methods according to the invention, as described herein, may be computer-implemented methods and may be used, where appropriate, for the delivery of electrical stimulation impulses for non-medical, or non-clinical, purposes, for example muscle conditioning.

The present invention may be used to treat or improve any muscular or neural condition that is alleviated through use of electrical stimulation.

The foregoing and other objects and advantages will appear from the descriptions that follow. In the description reference is made to the accompanying drawings, which forms a part hereof, and in which is shown by way of illustration specific principles of the control method in which the invention may be practiced. These principles will be described in sufficient detail to enable those skilled in the art to practice the invention, and it is to be understood that other principles may be utilized and that structural changes may be made without departing from the scope of the invention, for example, modifications in algorithms.

The method and apparatus described in the present invention enables the delivery of realistic sensory feedback to a user through electrical stimulation triggered by external actuators. The apparatus described in the present invention could be actuator devices transmitting an infrared stream. The apparatus described in the present invention could be Neuromuscular Electrical Stimulators (NMES) with IR sensing capability reacting to the actuator stream. The apparatus described in the present invention can connect to a remote central unit, optionally a mobile device, using the Bluetooth Low Energy (BLE) area network technology. The method described in the present invention uses novel encoding to transmit data between the different apparatus by means of IR, BLE, or any other communication. Certain embodiments described in the present invention enable the re-enactment of natural sensory perception. The present invention may be used for gaming and entertainment purposes. The present invention may also be used for training and learning purposes.

### BRIEF DESCRIPTION OF THE DRAWINGS

FIG. 1 shows a system including a plurality of receiver devices, a transmitter device, and a mobile device, according to the present invention.

FIG. 2 shows a custom infrared communications protocol in accordance with an embodiment of the invention.

### DETAILED DESCRIPTION

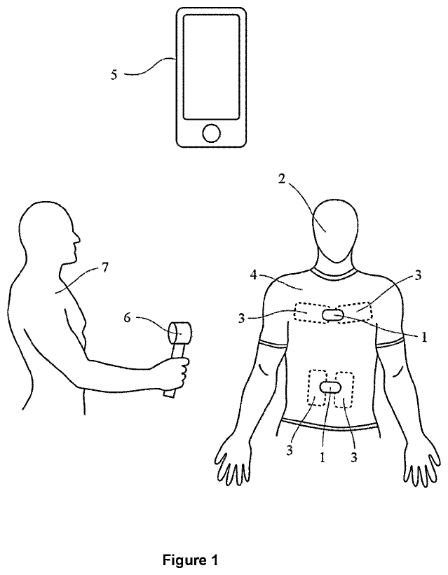

FIG. 1 shows an embodiment of the present invention where several NMES devices (1) are attached to an individual (2) by means of electrodes (3) rendered into a shirt (4). The NMES devices are wirelessly connected to a mobile phone (5) running a dedicated dashboard mobile application controlling the intensity of the NMES devices. The electrical stimulation delivered by the NMES devices is triggered by an actuator (6) controlled by another individual (7).

In the embodiment depicted, the mobile phone/mobile device (5) controls the intensity of the electrical stimulation delivered by the NMES devices (may also be referred to herein as receiver devices), and the actuator, in this case an infrared transmitter (may also be referred to herein as a transmitter device) (6) triggers the start and end of stimulation. In alternative embodiments, the NMES devices are instructed fully and triggered by the IR actuator alone, and the mobile device acts purely as a recording and/or display device. In yet further alternative embodiments, the mobile device controls the intensity of electrical stimulation and any other characteristics of the electrical stimulation applied by the NMES devices, for example the type of stimulation applied (e.g. could instruct the stimulation to ramp upwards in intensity from one value to another value over a fixed time period). In other words, the characteristics of the electrical stimulation can stem entirely from the mobile device, entirely from the transmitter device, or from a mixture of the two. The transmitter device does, however, in all embodiments, instruct the beginning of the electrical stimulation with its directed wireless communication received at the receiver device.

The receiver devices (1) comprise conductive media (3) in the form of pad electrodes woven into the fabric of the garment (4), in this case a t-shirt. The electrodes could be attached to the garment in any other way which limits movement of the electrodes relative to the body, such that the electrodes are positioned to stimulate the correct muscle groups. The receiver devices (1) also comprise a receiver (not depicted), configured to receive a directed wireless signal. As has already been described, the receiver may be configured to receive a directed wireless signal in a number of ways. The receiver may be configured to register angle of incidence of the incoming communication signal and to disregard signals arriving at an angle greater than a certain threshold. Alternatively, or additionally, the receiver may include a housing, for example a short tubular housing, configured to allow only signals arriving from certain directions. The receiver may be configured only to receive communications transmitted from direct line-of-sight by being configured as a receiver of wavelengths only received via direct line-of-sight. For example, configuring the receiver as an IR receiver limits it to receiving direct line-of-sight communication because IR is blocked by solid objects such as walls. A visible light receiver would be configured as a direct line-of-sight receiver for the same reasons.

The receiver devices (1) also comprise a processor configured to interpret received signals, which may include decoding and/or demodulating, and configured to initiate electrical stimulation via the electrodes based on the interpreted signal. The processor may be any processing unit capable of handling the inputs and outputs described herein. In this case, therefore, the processor must be capable of interpreting the received signal and instructing the electrical stimulation.

The receiver devices (1) may also comprise (not shown) electrical generation means with which to drive electrical current to the conductive media in order to stimulate the user. The electrical components on the receiver devices (1) may be powered by a portable power supply (also not shown), such as a battery, or could be connected to mains electricity if desired/required.

The receiver devices may be configured to provide haptic sensation via the electrodes. Haptic sensory feedback is the use of different actuators to create a realistic sensory response in order to enhance a user's experience in a virtual environment. Applications using haptic sensory feedback are very broad and cover among other things video gaming, entertainment, learning, physical training and clinical applications. Haptic sensory feedback is broadly defined as the sense of touch and includes vibration, texture, temperature, pain, force and proprioception sensations. In this way, the user can be provided with sensory haptic feedback based on electrical stimulation. The electrical stimulation can be triggered by a dedicated device by the means of IR communication or by an application running on a mobile device that is connected to the electrical stimulator unit by Bluetooth low energy (BLE) communication.

The different haptic feedback effects reproduced by the electrical stimulation are intended to provide the user with a more realistic immersive experience, or improved clinical experience. A wide range of haptic feedback can be provided by the invention including, but not limited to, physical activity such as lifting weight, feeling natural effects such as wind or rain, feeling the impact of different weapons. The scope of application is also wide and encompasses gaming, learning, military training as well as medical applications.

In the depicted embodiment, the transmitter device (6) comprises a processor configured to encode an electrical stimulation program into a wireless signal. In embodiments of the invention, however, the transmitter device (6) may not require a processor if used simply to initiate an electrical stimulation instructed by a third device (e.g. mobile device (5)). The encoding of the electrical stimulation program into a wireless signal by the processor of the transmitter device (6) will be discussed in greater detail below.

The transmitter device (6) also comprises a directional transmitter, configured to transmit the signal in one or more specific directions and not in others. The transmitter can be so configured in a number of ways. The signal generator itself may be configured only to generate a signal in a certain direction, for example by being directed by an antenna or other similar signal pathway. Additionally or alternatively, the transmitter may comprise a housing as shown in FIG. 1, the housing being made of a material which blocks transmission therethrough. For example, an opaque tubular housing could be used to block visible or IR communication other than from the end of the housing. A number of openings could be included in a housing to allow communication in more than one specific direction. Any communication from a directional transmitter as defined herein is a directed wireless communication.

The housing of the transmitter device (6) may also comprise a handle as shown.

The third device (5), in the depicted embodiment a mobile device, is configured for wireless communication with both the transmitter device (6) and receiver devices (1). The communication protocol used is Bluetooth Low Energy, but may be any other communication protocol. Advantageously, the communication protocol used by the third device (5) should be different to that used for communication between the transmitter and receiver devices.

The third device (5) comprises a processor configured to store received data relating to the electrical stimulation applied by the electrodes (3) and from the transmitter device (6) relating to the transmitted signal. Using these data, the third device (5) can compare the transmitted signal to the applied stimulation, thereby checking whether the signal was received at the receiver device, and checking whether the stimulation was applied correctly given the transmitted signal. The third device may also comprise storage means configured to store the data received for retrieval at a later time. Data relating to the electrical stimulation applied by the conductive medium may comprise a length of time of applied stimulation, electrical characteristics of the stimulation (e.g. voltage, current, etc.), number of electrodes stimulated, location of stimulated electrodes, and any other characteristic of the stimulation deemed useful. Similarly, data relating to the transmitted signal may comprise a time of transmission, content of transmission, characteristics of the encoding such as carrier frequency, and any other characteristic of the transmission deemed useful.

One such instance of data retrieval could be initiated by a user via a user interface on the third device (5). A user could request a listing of stimulations applied within, for example, the past week. The third device may be configured to retrieve and display such a list from storage in response to the request. The display and user interface may be one and the same, for example in the case of a touch-screen display.

Communications Between Transmitter and Receiver

Communication between transmitter and receiver may be directional by using a directional transmitter or directional receiver, as described above. The communication may be modulated to avoid crosstalk and ambient sources.

In one embodiment of the invention, infrared light is used as the communication medium between transmitter and receiver devices.

Infrared (IR), sometimes called infrared light, is electromagnetic radiation (EMR) with wavelengths longer than those of visible light. IR is generally considered to encompass wavelengths from approximately 1 millimeter (300 GHz) to the nominal red edge of the visible spectrum, at approximately 700 nanometers (frequency 430 THz).

Embodiments of the invention use IR light as a wireless mobile technology for device communication over short ranges.

Because of IR's limitations, such as requiring line-of-sight, inability to penetrate objects, and short transmission range, communication interception is difficult. Thus, data transmitted between IR devices may not need to be encrypted.

IR light-emitting diodes (LED) are used to transmit IR signals, which pass through a lens and focus into a beam (i.e. directional stream) of IR data. The use of focussing lenses to provide directional communication is not, of course, limited to IR in the context of the present invention. The beam source is rapidly switched on and off for data encoding.

The IR beam data is received by an IR device equipped with a silicon photodiode. This receiver converts the IR beam into an electric current for processing. Because IR transitions more slowly from ambient light than from a rapidly pulsating IR signal, the silicon photodiode can filter out the IrDA signal from ambient IR.

IR transmitters and receivers are classified as directed and non-directed. A transmitter or receiver that uses a focused and narrow beam is directed, whereas a transmitter or receiver that uses an omnidirectional radiation pattern is non-directed. Some level of directionality of communication can be achieved by using a directed transmitter or directed receiver with non-directed receiver and non-directed transmitter, respectively, however, use of a directed transmitter with directed receiver is preferred.

The invention provides electrical stimulation devices featuring an IR receiver and actuator devices featuring an IR transmitter. The electrical stimulation devices can be attached to a garment with rendered carbon electrodes, an accessory such as a strap, sleeve or wrap with rendered carbon electrodes or to hydrogel electrodes. The transmitter device can be in the form of a handled remote control, a weapon replica, a central unit or any other forms. The IR beam generated by the transmitter device must be directed toward the electrical stimulator unit The IR emitter can be an IR LED with a wavelength comprised between 780 nanometers and 1000 nanometers. It could also be a laser IR LED when a narrower beam is needed. The IR receiver consists of a photo detector sensitive to IR light.

Both the IR transmitter and receiver devices can be connected to a mobile application through a Bluetooth interface to record and display the different events triggered by the IR actuator devices and the response of the IR electrical stimulator devices.

Infrared communication is among the simplest wireless communication methods, and it serves as a cost-effective way of transmitting a small number of bits of data, wirelessly. Infrared LEDs produce light not visible to the human eye. Typically, the wavelength of light that such devices output is around 950 nanometers. However, IR LEDs aren't the only thing that can emit IR or near IR waves. Many other sources, like light bulbs and the sun itself, release IR waves, which is one of the difficulties when dealing with IR communications.

Furthermore, anyone can send IR signals. Typically, no handshake, authentication, or authorization takes place between the sender and the receiver. A television, for example, can be controlled by any remote using the same protocol.

The simplest method for transmitting binary values with an IR LED would be to turn the infrared LED on (to send a logical 1) or to leave it turned off (which could represent a logical 0) for a certain period. The sender could do this until all the data bits have been transmitted. Unfortunately, this is not ideal since many other sources emit IR radiation. The receiver may not be able to filter out unwanted signals from other sources.

To overcome this issue, the sender is required to pulse the LED on and off very quickly, instead of just turning it on and off. Typically, a frequency of 38,000 Hz is used, and this is also referred to as the carrier frequency of the IR signal. Note that other wavelengths and carrier frequencies are also possible—for example, 940 nm and 36 kHz, or any other as will be apparent to the skilled person. In any case, most IR receivers will function with slight variations in wavelength and carrier frequency.

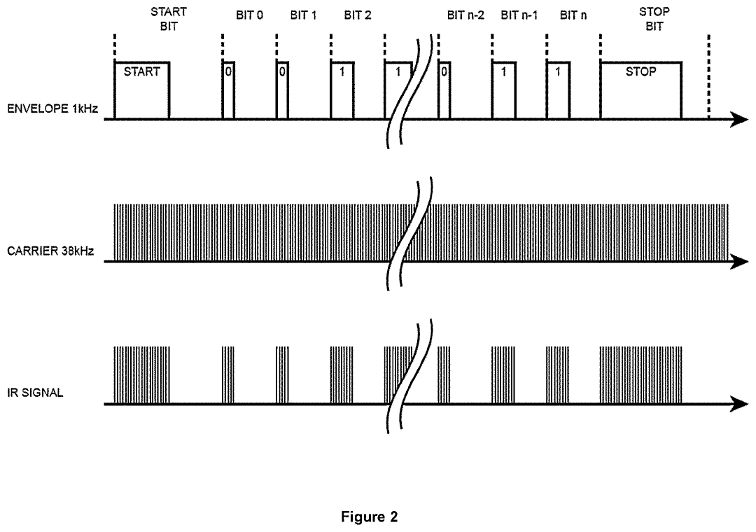

FIG. 2 depicts a custom IR protocol based on pulse width modulation (PWM). The carrier frequency is set at 38,000 Hz with a duty cycle of 25%. The frequency of the envelope PWM signal is set at 1,000 Hz. The IR data frame starts with a START bit followed by a variable number of data bits. It ends with a STOP bit. The START bit consists of the activation of the carrier frequency during 1,000 microseconds followed by 1,000 microseconds of inactivity. A bit of value zero consists of the activation of the carrier frequency during 210 microseconds followed by 790 microseconds of inactivity. A bit of value one consists of the activation of the carrier frequency during 420 microseconds followed by 580 microseconds of inactivity. The STOP bit consists of the activation of the carrier frequency during 1,500 microseconds followed by 500 microseconds of inactivity. FIG. 2 represents a possible data frame. The envelope signal contains the data to be transmitted. The IR signal is the result of the carrier frequency being activated when the envelope signal is on.

It will be appreciated that while specific values are given above for envelope frequency, carrier frequency, one and zero activations, duty cycle, and start and end activations, that these may be adjusted as the skilled person sees fit. That said, it is important for data clarity that certain values remain different to one another, for example, start, stop, one, and zero activation times should remain different in order to distinguish therebetween. Activation time may also be referred to as pulse width.

The IR receiver will demodulate the IR signal and retrieve the envelope data frame. This data frame can contain information such as the identification of the transmitter device, the haptic feedback to trigger, the intensity of the electrical stimulation and the delay after which the haptic feedback should be triggered among other things.

It will be appreciated that although IR has been used to demonstrate the custom communication protocol in this example, that other directional wireless communications protocols are suitable. For example, visible light can be used in darker environments or with receivers particularly effective at removing ambient sources of light. Visible light may also be suitable at relatively short distances with increased intensity of signal. Laser light is also suitable. Visible light may also be modulated in a similar manner to that described in relation to FIG. 2.

It is also possible to use radiofrequency (RF) and Bluetooth as directional communications protocols, for example with the use of antennas, and presently used in location finding technology (which, of course, also relies on directional communication). Although specific examples have been given herein, any and all directional communication is suitable for use with the present devices, systems, and methods. IR communication is, however, preferred, for reasons given above.

### Embodiments

1. An apparatus for providing electrical stimulation to different muscle and nerve groups.

2. The apparatus of Embodiment 1 wherein the electrical stimulation is delivered to the stimulated object (individual) by means of wireless NMES devices.

3. The apparatus of Embodiment 1 wherein the electrical stimulation delivered to the stimulated object (individual) enable haptic sensory feedback.

4. The apparatus of Embodiment 1 wherein the electrical stimulation delivered to the stimulated object (individual) is triggered by Infrared actuator devices.

5. The apparatus of Embodiment 1 wherein the electrical stimulation is delivered to the stimulated object (individual) by means of electrodes or electrically conductive materials rendered into garments or accessories.

6. The apparatus of Embodiment 1 wherein the intensity of the electrical stimulation can be controlled by either a mobile application using Bluetooth interface or by an Infrared actuator device.

7. An apparatus to trigger electrical stimulation delivered by the apparatus of Embodiment 1 by means of an Infrared communication interface.

8. The apparatus of Embodiment 7 wherein the Infrared interface protocol consists of several encoded types of sensory feedback.

9. An apparatus for attaching one or multiple apparatus of Embodiment 1 to the stimulated object (individual).

10. The apparatus of Embodiment 9 whereby the electrical stimulation is delivered by means of electrodes embedded into a garment or an accessory designed on that purpose.

11. A method for encoding different types of sensory feedback.

12. The method of Embodiment 11 wherein one or multiple apparatus connect to the same mobile application.

1. A device for providing electrical stimulation to a user, comprising:
a conductive medium configured to apply electrical stimulation to a user;
a receiver, configured to receive a directed wireless signal; and
a processor, configured to:
interpret the received signal; and
initiate electrical stimulation via the conductive medium based on the interpreted signal.

2. The device according to claim 1, wherein the conductive medium is embedded within a garment, optionally wherein the garment comprises a strap, sleeve, wrap, or item of clothing.

3. The device according to claim 1, wherein the conductive medium comprises an electrode, optionally a carbon electrode or a hydrogel electrode.

4. The device according to claim 1, wherein the conductive medium is configured to apply an electrical stimulation causing a haptic sensation in the user and/or wherein the interpreted signal is configured to instruct an electrical stimulation causing a haptic sensation in the user.

5. The device according to claim 1, wherein the receiver is configured to receive signals from certain directions and not from others.

6. The device according to claim 1, wherein the receiver is configured to receive the directed wireless signal only when the receiver has direct line-of-sight of a transmitter of the directed wireless signal.

7. A device for transmitting an electrical stimulation program to a device configured to provide electrical stimulation to a user, the transmission device comprising:
a processor, configured to encode an electrical stimulation program into a wireless signal; and
a directional transmitter, configured to transmit the signal in one or more specific directions and not in other directions.

8. The device according to claim 7, wherein encoding the electrical stimulation program into the signal comprises:
receiving the electrical stimulation program;
applying an communication protocol to the electrical stimulation program using pulse width modulation.

9. The device according to claim 8, wherein applying a communication protocol to the electrical stimulation program using pulse width modulation comprises:
selecting a carrier frequency known to both the device for transmitting the electrical stimulation program and to the device configured to provide electrical stimulation to a user;
selecting a start pulse width and an end pulse width known to both the device for transmitting the electrical stimulation program and to the device configured to provide electrical stimulation to a user, wherein the start and end pulse widths are not equal;
selecting a first pulse width to define a bit value of one and a second pulse width to define a bit value of zero, wherein the first and second pulse widths are not equal to one another or to the start and end pulse widths;
converting the bits of the electrical stimulation program into pulses;
concatenating the start and end pulses to the beginning and end, respectively, of the converted electrical stimulation program; and
applying the carrier frequency to the concatenated pulses.

10. The device according to claim 7, wherein the electrical stimulation program comprises at least one of: an identifier for the device for providing electrical stimulation to a user, an identifier for transmitting an electrical stimulation program, a length of an electrical stimulation, an intensity of electrical stimulation, a type of electrical stimulation, and a delay after reception of the signal before electrical stimulation begins.

11. The device according to claim 10, wherein the electrical stimulation program is configured to instruct an electrical stimulation causing a haptic sensation in the user.

12. A system, comprising:
a first device for providing electrical stimulation to a user according to claim 1;
a second device for transmitting an electrical stimulation program according to claim 7.

13. The system according to claim 12, further comprising:
a third device, in communication with the first and second devices via a wireless communications protocol, the third device comprising a processor configured to:
receive, from the first device, data relating to the electrical stimulation applied by the conductive medium; and
receive, from the second device, data relating to the transmitted signal.

14. The system according to claim 13, wherein the third device comprises a display and is configured to record and display the data relating to the transmitted signal and/or the data relating to the electrical stimulation applied by the conductive medium.

15. The system according to claim 12, wherein the wireless communications protocol is Bluetooth Low Energy.

16. A method for providing electrical stimulation to a user, comprising:
transmitting an electrical stimulation program from a device according to claim 7;
receiving the electrical stimulation program at a device according to claim 1; and
providing electrical stimulation to the user according to the received electrical stimulation program.

17. A method for providing electrical stimulation to a user, comprising operating the system of claim 12.

18. A computer-implemented method for encoding an electrical stimulation program into an encoded wireless signal, comprising:
selecting a carrier frequency;
selecting a start pulse width and an end pulse width, wherein the start and end pulse widths are not equal;
selecting a first pulse width to define a bit value of one and a second pulse width to define a bit value of zero, wherein the first and second pulse widths are not equal to one another or to the start and end pulse widths;
converting the bits of the electrical stimulation program into pulses;
concatenating the start and end pulses to the beginning and end, respectively, of the converted electrical stimulation program; and
applying the carrier frequency to the concatenated pulses.

19. The device according to claim 1, wherein the directed wireless signal is a directed infrared signal.
